(19) United States
(12) Patent Application Publication
(10) Pub. No.: US 2020/0391006 A1
(43) Pub. Date: Dec. 17, 2020

(54) CATHETER TUBING SYSTEM

(71) Applicant: CIC Fund Securitisation S.A., Luxembourg (LU)

(72) Inventors: Deneen T PLESSALA, South Mobile, AL (US); Matthew G MCINTYRE, Theodore, AL (US); Peter T FALKNER, Mobile, AL (US)

(21) Appl. No.: 16/971,812

(22) PCT Filed: Feb. 22, 2019
(86) PCT No.: PCT/US2019/019157

§ 371 Date: Aug. 21, 2020

Related U.S. Application Data
(60) Provisional application No. 62/633,951, filed on Feb. 22, 2018.

(51) Int. Cl. A61M 25/00; A61M 1/00; A61M 39/10

(52) U.S. Cl. CPC A61M 25/0017; A61M 1/0086; A61M 1/0001; A61M 2039/1088; A61M 39/10; A61M 2039/1083; A61M 25/0026

(57) ABSTRACT

A tubing assembly device for use in combination with a typical Foley or multi-lumen urinary catheter that assists in the prevention of said tubes becoming entangled or mismanaged by the physician or patient includes a drainage tube and at least one fluid delivery tube having connectors operable to attach to catheter lumens and container lumens. The tubes are bundled together by having a portion of their exterior surface fixedly attached to a portion of the exterior surface of another tube. In order to prevent kinks where the device is attached to the catheter, the upper portions of each tube are necessarily longer than the lower portions. Also, as a further option, the connectors on opposing ends of the tubes include a variety of markings to ensure proper attachment.

### RELATED APPLICATIONS

This application claims priority to U.S. Provisional Application No. 62/633,951 filed 22 Feb. 2018. The entire contents of the above application are hereby incorporated by reference as though fully set forth herein.

### FIELD

The present invention relates in general to catheter tubing. More specifically, the present invention relates to a multi-tube catheter tubing assembly device for a urinary catheter.

### BACKGROUND

It is well known in the art that indwelling urinary catheter systems, such as a Foley catheter consists of two lumens: (i) one for drainage of urine from the bladder and (ii) a second lumen used to inflate the retention balloon that lies within the bladder to hold the catheter in place. A triple lumen catheter currently in the art has an additional lumen for instillation of fluid into the bladder. In place of the third lumen used to instill fluid, this optional lumen may be used in order to continuously efflux fluid and irrigate the periurethral space and the catheter body to prevent formation of biofilm and further ensuing bacterial infection. The fluid instilled generally includes certain therapeutic agents used in intravesical therapy such as immunotherapy agents or chemotherapeutic agents, antispasmodic agents and numbing agents, such as lidocaine. The fluid effluxed generally includes antiseptics, antibiotics or antimicrobials and/or combinations thereof to prevent biofilm formation on the exterior surface of the catheter body. Such a catheter system is more fully described in patent application number PCT/US17/26450, the contents of which are incorporated by reference herein.

While the use of a multi-lumen tubing system is preferred, present systems known in the art utilize long separate catheter tubes that connect each catheter lumen to its opposing end source, whether it be the drainage container for the drainage lumen or the fluid container used to instill fluid or irrigate the periurethral space and catheter body. Often, these separate tubes have a tendency to tangle with themselves and the patient's body and become cumbersome for the patients and the medical staff while treating the patient. It is therefore an object of the present invention to provide a catheter tubing device assembly to reduce tangling with other tubing or the patient's body.

### BRIEF SUMMARY OF THE INVENTION

The preferred embodiment of the present invention comprises a drainage tube and a fluid delivery tube, each having an exterior surface and connectors on both its ends wherein the tubes are bundled together by having a portion of the exterior surface of the drainage tube is fixedly attached to a portion of the exterior surface of said fluid delivery tube. In the case of multiple fluid delivery lumens, the present invention anticipates having a plurality of fluid delivery tubes wherein a portion of the exterior surface each tube is fixedly attached to the exterior surface of another tube in the assembly. The connector on the first end of the drainage tube is operable to attach to a drainage lumen of a urinary catheter, and the connector on the second end is operable to attach to a container for holding the drained fluid. The connectors on the first end of the fluid delivery tube(s) are operable to attach to a fluid delivery lumen of a urinary catheter, and the connector on the second end is operable to attach to a container assembly for holding the fluid to be delivered. As an additional option, the inside diameter of the drainage tube may be greater than the diameter of any fluid delivery tube. Similarly, the upper portion of the tubes between the catheter and attachment portion is necessarily longer to prevent kinks in the tubing.

### BRIEF DESCRIPTION OF THE DRAWINGS

FIG. 1 is a side view of the catheter tubing device assembly of the present invention showing a drainage tube and a plurality of fluid delivery tubes.

FIG. 2 is a side view of the catheter tubing device assembly of the present invention attached to a multi-lumen urinary catheter.

### DETAILED DESCRIPTION

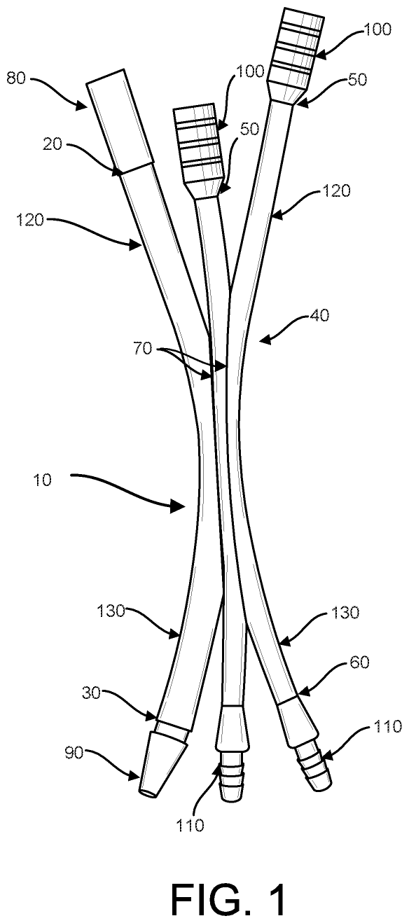

Turning to FIG. 1, the catheter tubing assembly device of the present invention is shown in detail and comprises a drainage tube 10 having an exterior surface with a first end 20 and a second end 30, and a plurality of fluid delivery tubes 40 each having an exterior surface, a first end 50, and second end 60. In order to prevent entanglement of these tubes, an attachment portion 70 of the exterior surface of the drainage tube is fixedly attached to a portion of the exterior surface of the fluid delivery tube 40 such that the tubes form a bundle. The attachment portion 70 may be prefabricated such that the assembly constitutes a contiguous body or the individual tubes can be separately bonded together using bonding means known in the art, e.g. adhesives, glues, or epoxies. Although not shown in the figures, for this patent, the present invention only requires at least one drainage tube 10 and at least one fluid delivery tube 40, to be compatible with a typical triple lumen urinary catheter. Moreover, as shown in FIG. 1, the drainage tube diameter, i.e. the inner diameter of the tube, is necessarily larger than the inner diameter of the fluid delivery tubes.

The drainage tube 10 has a first drainage tube connector 80 at its first end 20 and a second drainage tube connector 90 at its second end 30. The fluid delivery tube 40 has a first delivery tube connector 100 at its first end 50, and a second fluid delivery tube connector 110 at its second end 60 operable to attach to a container assembly known in the art for holding fluid that is to be delivered.

Based on the multi-lumen design of the catheter, in order to avoid kinks or looping in the entire catheter tubing system. the upper portions 120 of each tube in the assembly, i.e. the length of tubing between the first ends 20, 50 and the attachment portion 70, is necessarily longer than lower portions 130 of each tube, i.e. the length of tubing between the second ends 30, 60 and the attachment portion 70.

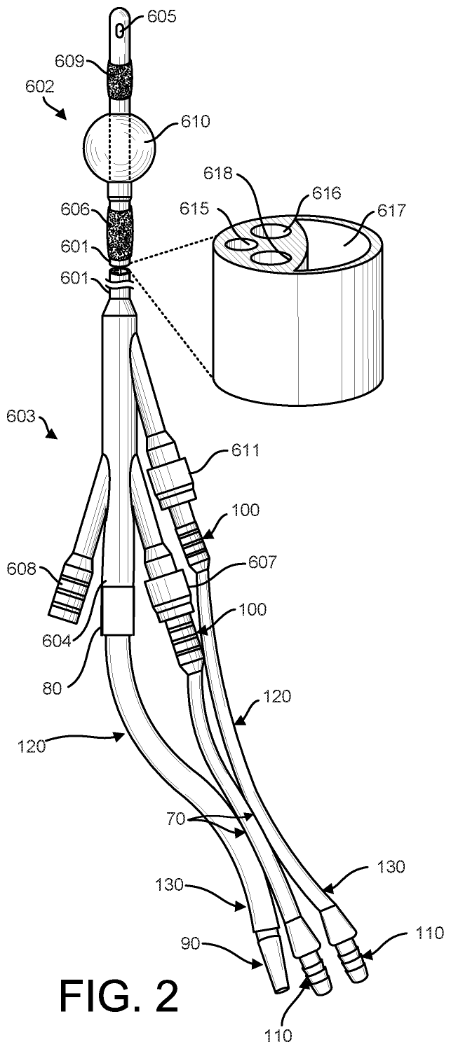

Turning to FIG. 2, the embodiment of the invention displayed in FIG. 1 is shown being used with a 3-way catheter as described in patent application number PCT/US17/26450. This catheter is merely illustrative of the general components of a 3-way catheter known in the art and how the terminal ends of such a catheter are used in combination with the subject catheter tubing assembly device. An elongated tubular catheter body 601 having a distal end 602 and a proximal end 603 is shown. A drainage lumen 604 extends through tube 617 in the catheter body 601 from the distal end 602 to the proximal end 603, and the drainage lumen 604 communicates with an opening or eyelet 605 in the catheter body 601 at the distal end 602 of the catheter body 601 through which the fluid may flow into the drainage lumen 604 when the catheter is used to drain a fluid from a cavity, duct, or vessel (e.g., draining urine from a person's bladder). A first sleeve portion 606 constructed from a semipermeable membrane is formed over the catheter body 601. A fluid delivery lumen 607 extends from the catheter body 601 at the distal end 602. The fluid delivery lumen 607 connects with the first sleeve portion 606 using tube 616 that runs through the length of the catheter body 601. The fluid delivered into the catheter body 601 through the tube may be continuously effluxed from the sleeve portion 606 through the semipermeable membrane in a circumferential controlled delivery to continuously irrigate the periurethral space and the catheter body 601 to prevent formation of biofilm and further ensuing bacterial infection. However, the sleeve portion 606 is not required as the fluid may be used to continuously or intermittently irrigate the bladder using a traditional 3-way Foley catheter.

A second sleeve portion 609 constructed from a semipermeable membrane is formed over the catheter body 601 above the tube section 610. A second fluid delivery lumen 611 extends from the catheter body 601 at the distal end 602. The fluid delivery lumen 611 connects with the sleeve portion 609 using tube 618 that runs through the length of the catheter body 601. The fluid delivered into the catheter body 601 through the tube 618 is continuously effluxed from the sleeve portion 609 through the semipermeable membrane and into the bladder itself.

The first drainage tube connector 80 is operable to attach to the cylindrical drainage lumen 604 by using attachment means known in the art, including using an open-ended cylindrical cap, as shown in the figure, that is force fitted over the drainage lumen 604. The second drainage tube connector 90 is operable to attach to a container assembly known in the art for holding fluid that is drained by using attachment means known in the art, including using an open-ended conically shaped cap, and shown in the figure, that is force fitted within the inlet lumen for the drainage container (not shown).

The first fluid delivery tube connector 100 is operable to attach to the terminal ends of the fluid delivery lumens 607, 611 and the second fluid delivery tube connector 110 is operable to attach to a container assembly known in the art for holding fluid that is to be delivered (not shown). The first and second delivery tube connectors 100, 110 are selected from a group of pressurized connectors known in the art, including but not limited to male/female locking mechanisms, such as Leur locks. The terminal ends of the fluid delivery lumens 607, 611 for a urinary catheter may be male or female. In FIG. 2, the first delivery tube connector 100 comprises a female connector that attaches to the male terminal end of the fluid delivery lumen 607, 611 and the second fluid delivery tube connector 110 comprises a male connector that attaches to the inlet lumen for fluid container (not shown).

Obviously, the bundling of multiple fluid delivery tubes 40 makes it difficult on the user to make sure that the connectors 100, 110 of each tube are attached to the proper catheter lumen and fluid delivery container. In order to prevent such a problem, the present invention optionally may include a marking system on its connectors 100, 110 so that the user can easily distinguish the connectors 100, 110 of one fluid delivery tube 40 from another when making necessary connections. Such markings on the connectors may be made by using different colors, alternative fitting configurations, or different inscriptions on the fitting connector itself, or any other comparable marking mechanism known in the art.

Although not shown in the figures, the invention anticipates having sufficient length and space on each individual fluid delivery tube 40 to accommodate a roller clamp. These clamps are well known in the art and are used to adjust the volumetric flow of fluid through the catheter by adjustably clamping the flexible catheter tubing. For the present invention, the roller clamp would be positioned around an exterior portion of the tube either before or after the attachment portion 70 of the tube 40 that is fixedly attached to another fluid delivery tube 40 or drainage tube 10.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

1) A catheter tubing device assembly comprising:
a drainage tube having an exterior surface, a first end, and a second end;
a first drainage tube connector and a second drainage tube connector, wherein said first end of said drainage tube is attached to said first drainage tube connector, and said second end of said drainage tube is connected to said second drainage tube connector;
a fluid delivery tube having an exterior surface, a first end, and a second end; and
a first fluid delivery tube connector and a second fluid delivery tube connector, wherein said first end of said fluid delivery tube is attached to said first fluid delivery tube connector, and said second end of said fluid delivery tube is connected to said second fluid delivery tube connector;
wherein a portion of the exterior surface of said drainage tube is fixedly attached to a portion of the exterior surface of said fluid delivery tube to form an attachment portion such that said drainage tube and said fluid delivery tube form a bundle.

2) The device assembly of claim 1 wherein the first drainage tube connector is operable to attach to a drainage lumen of a catheter, and the second drainage tube connector is operable to attach to a container assembly operable to hold fluid that is drained.

3) The device assembly of claim 1 wherein the first fluid delivery tube connector is operable to attach to a fluid delivery lumen of a catheter, and the second fluid delivery tube connector is operable to attach to an assembly holding fluid to be delivered.

4) The device assembly of claim 3 wherein the connectors are pressurized male/female Luer locks.

5) The device assembly of claim 1 wherein the drainage tube further comprises a drainage tube diameter, wherein the fluid delivery tube further comprises a fluid delivery tube diameter, and wherein said drainage tube diameter is larger than said fluid delivery tube diameter.

6) The device assembly of claim 1 wherein each tube in the assembly device further comprises an upper portion between the first end and the attachment portion and a lower portion between the second end and the attachment portion.

7) The device assembly of claim 6, wherein a length of the upper portion of each tube in the assembly is greater than a length of the lower portion of each tube in the assembly.

8) The device assembly of claim 1, further comprising a plurality of fluid delivery tubes, wherein each fluid delivery tube of said plurality of fluid delivery tubes comprises an exterior surface, a first end, a second end, and a pair of fluid delivery tube connectors; wherein the first end of each fluid delivery tube is attached to a fluid delivery tube connector, and the second end of each said fluid delivery tube is connected to a fluid delivery tube connector.

9) The device assembly of claim 8, wherein a portion of the exterior surface of each of said drainage tubes and said plurality of fluid delivery tubes is fixedly attached to the exterior surface of at least one other tube of said drainage tube and said plurality of fluid delivery tubes to form an attachment portion.

10) The device assembly of claim 9 wherein each of said tubes in the assembly further comprises an upper portion disposed between the first end and the attachment portion and a lower portion between the second end and the attachment portion.

11) The device assembly of claim 10 wherein the length of the upper portion of each tube in the assembly is greater than the length of the lower portion of each tube in the assembly.
